(19) United States
(12) Patent Application Publication
(10) Pub. No.: US 2003/0187369 A1
(43) Pub. Date: Oct. 2, 2003

(54) Optical pullback sensor for measuring linear displacement of a catheter or other elongate member

(76) Inventors: Stephen B. Lewis, Folsom, CA (US); Michael J. Eberle, Fair Oaks, CA (US)

Correspondence Address: LEYDIG VOIT & MAYER, LTD, TWO PRUDENTIAL PLAZA, SUITE 4900, 180 NORTH STETSON AVENUE, CHICAGO, IL, 60601-6780, US

(21) Appl. No.: 10/109,191

(22) Filed: Mar. 28, 2002

(51) Int. Cl. A61B 5/103

(52) U.S. Cl. 600/587

(57) ABSTRACT

An optical pullback sensor assembly is disclosed that facilitates measuring displacement of a flexible elongate member, such as an ultrasound catheter probe that is inserted within a body. The disclosed embodiments of the optical pullback sensor assembly include a light source that illuminates a target surface that reflects at least a portion of light received from the light source. An optical sensor array including a set of sensor cells is arranged opposite the target surface to receive light reflected by the target surface. The optical sensor array is oriented to render image frames that are utilized to determine displacement of the flexible elongate member. A tracking guide on the pullback sensor assembly confines relative movement between the target surface and the optical sensor array in a direction of measured displacement of the optical sensor array. The measured displacement of the target surface in relation to the optical sensor represents displacement of the flexible elongate member.

### AREA OF THE INVENTION

The present invention generally relates to the area of diagnostic medical equipment, and more particularly to diagnostic devices for locating and/or treating problematic blockages within coronary arteries by means of a sensor and/or treatment components mounted proximate the distal end of a flexible elongate member such as a catheter or guide wire. In particular, the present invention relates to devices enabling measurement of the linear displacement of a flexible elongate member, such as a catheter, within a vasculature.

### BACKGROUND OF THE INVENTION

The focus of innovation in diagnosing cardiovascular disease has migrated from external imaging processes to internal, catheterization-based, diagnostic processes. Diagnosis of cardiovascular disease has been performed through angiogram imaging wherein a radiopaque dye is injected into a vasculature and a live x-ray image is taken of the portions of the cardiovascular system of interest. Magnetic resonance imaging (MRI) has also been utilized as well. More recently, however, diagnostic equipment and processes have been developed for diagnosing vasculature blockages and other circulatory disease by means of ultra-miniature sensors placed upon a distal end of a flexible elongate member such as a catheter, or a guide wire used for catheterization procedures.

One such type ultra-miniature sensor is an intravascular ultrasound (IVUS) imaging device mounted upon the distal end of a catheter. An example of such imaging device is provided in Proudian et al. U.S. Pat. No. 4,917,097. Such sensors, utilized to create a visual image from within a blood vessel facilitate locating and determining the severity of stenoses or other disruptions of blood flow within the vessels of the human body. Such devices are commonly used to identify plaque buildup within blood vessels, position a catheter for treatment (e.g., positioning a balloon during a balloon angioplasty procedure), and determine the effectiveness of the treatment procedure by rendering images of the vessel based upon received echoes from within the vessel.

At an early stage, IVUS imaging comprised displayed transverse cross-sectional images of a vessel as an imaging probe was moved relative to the length of the vessel under investigation. Initially, the act of moving the imaging probe was performed by hand. Such method is satisfactory for identifying occlusions within a vessel. However, as IVUS imaging systems advanced, a linear dimension was added to IVUS imaging. A composite, sagittal cross-sectional image was created from multiple cross-sectional images taken at measurable positions within the length of a vessel. In such imaging, it is important to accurately track the position of the IVUS device within the vessel under investigation to properly gauge both the positions and span of occlusions for which treatment (e.g., placement of a stent) is needed.

Controlled movement of the IVUS imaging probe in relation to a vessel has been achieved previously by means of mechanical pullback devices that are operated by hand or alternatively by means of a pedal-controlled electric motor. An example of such a device is provided in Vujanic et. al. U.S. Pat. No. 6,290,675. Previously, in such pullback devices, tracking movement of an IVUS catheter in relation to a vessel is achieved by monitoring the movement of a driving mechanism (e.g., a screw-driven roller) and correlating that movement to rotation of a driving roller that, in turn, linearly displaces a catheter.

Alternatively, linear displacement of a catheter is tracked in Van Egmond et al. U.S. Pat. No. 5,709,661, via a slotted wheel that shares a rotating axis with a drive roller. The slots of the slotted wheel rotate through an LED emitter/sensor pair as the catheter is inserted or withdrawn. The LED sensor transmits a signal representing the number of slots that have passed through the sensor's beam path. The slot signal (in association with a direction signal) is translated into linear displacement of the catheter.

The known pullback devices utilize a pair of rollers to engage a catheter inserted within a patient's body. Linear displacement of the catheter is correlated to a measurable rotation of the rollers and/or other moving mechanical parts (a screw-drive and gears linked to a motor driven rotating axel) as the catheter is inserted into or withdrawn from a patient. Thus, accurate depiction of a vessel's sagittal cross-section requires precise correlation of catheter movement to the rotation of the rollers. However, the rollers can slip in relation to the catheter-especially when the catheter becomes wet. If the catheter slips in relationship to the rollers, then the length of the image becomes distorted at the points where the catheter slipped.

Another shortcoming of the prior known devices is the cost. The devices are used for a single patient and then discarded. Therefore, it is desirable to make such devices as inexpensively as possible. However the known devices, especially the motor-driven ones, have complex mechanical assemblies including motors and other costly components.

### SUMMARY OF THE INVENTION

The present invention comprises an improved pullback device including a linear displacement sensor that addresses the shortcomings of the above-described pullback devices. More particularly, the present invention facilitates reliable tracking of actual catheter movement. Eliminating the need for a drive motor also potentially reduces the cost of producing sensors embodying the present invention.

The present invention comprises an optical sensor array-based pullback device that facilitates tracking position/displacement of a flexible elongate member, such as a catheter, within a human body. The optical sensor array senses linear movement of the catheter by sensing light reflected in non-uniform patterns from a surface moving across the sensor's field of view. Linear shifting of unambiguous scatter patterns on the optical sensor array by reflected coherent collinear light facilitates tracking linear movement of the elongate member and thus facilitates creating accurate sagittal (i.e., longitudinal) cross-section images of blood vessels from a series of transverse cross-section images.

The present invention contemplates utilization of a light emitter/optical sensor array in a variety of modes to detect movement of a flexible elongate member in accordance with various embodiments of the present invention. Such variations described, by way of example, herein below with reference to a number of illustrative embodiments are considered within the scope of the present invention.

### BRIEF DESCRIPTION OF THE DRAWINGS

While the appended claims set forth the features of the present invention with particularity, the invention, together with its objects and advantages, may be best understood from the following detailed description taken in conjunction with the accompanying drawings of which:

FIG. 1 is an illustrative depiction of an optical pullback sensor assembly and catheter in accordance with an embodiment of the present invention;

FIG. 2 is a cross-sectional view taken along the length of the sensor assembly depicted in FIG. 1;

FIG. 3 is a drawing depicting an alternative embodiment of an optical pullback sensor assembly embodying the present invention;

FIG. 4 depicts an alternative optical pullback sensor assembly wherein displacement of the catheter is directly sensed by the optical sensor array; and

FIG. 5 depicts the optical pullback sensor array of FIG. 4 in a closed, operative mode;

FIG. 6 depicts an alternative optical pullback sensor assembly wherein the catheter is engaged by a hinged lid;

FIGS. 7, 8 and 9 depict alternative views of the optical pullback sensor assembly depicted in FIG. 6;

FIGS. 10 and 11 depict an exemplary system and environment within which the pullback sensor assembly is utilized; and

FIG. 12 depicts an exemplary set of steps performed to generate images from correlated cross-sectional ultrasound image data and imaging probe displacement data.

### DETAILED DESCRIPTION OF PREFERRED EMBODIMENTS

Before addressing illustrative embodiments of the inventions set forth in the figures, it is noted that the present invention relies upon an optical sensor array including multiple cells to sense the movement of a target surface within a field of view sensed by the optical sensor array. The optical sensor cells are oriented to render image frames to facilitate detection of catheter displacement in the direction of catheter travel. The target surface movement corresponds to linear displacement of a flexible elongate member suitable for insertion into the human body. Such optical sensor array technology is presently incorporated into known optical mice for positioning a pointer upon a graphical user interface display device. In accordance with embodiments of the present invention, a surface moves across the optical sensor array's field of view in an amount corresponding to linear displacement of a catheter. The sensor array output is processed to render an output signal corresponding to the linear displacement of the catheter as it is inserted and/or withdrawn from within a body. The displacement information is combinable, in an imaging system, with image data taken at measured positions of a vessel to render an image depicting a sagittal cross-section image of the vessel.

The present invention contemplates a variety of arrangements for measuring linear displacement of a catheter. In embodiments of the invention, the catheter itself is pulled within the field of view of the optical sensor array. Light strikes a surface of the catheter and scatters in a non-uniform pattern, based upon non-uniformities on the catheter surface, onto the sensor array. In a particular embodiment of the invention, a proxy material, such as a sleeve placed around the catheter at the portion that passes before the sensor array, is used as a target surface drawn past the field of view of the optical sensor array. The proxy (e.g., sleeve) moves a distance corresponding to the catheter displacement and advantageously provides a reflective surface having greater variations in comparison to the reflective surface of the catheter and enhances detection accuracy.

In an alternative embodiment, a patterned target surface is incorporated to enhance detection of the catheter's displacement. Rather than detect light reflected from a surface of the catheter (or catheter sleeve), a carriage fixedly holds the catheter and displacement of the catheter is sensed by light reflected off the patterned target surface and received by the optical sensor array. In embodiments of the invention, the patterned surface is located either on a platform over which the carriage (carrying the sensor array) slides or on a carriage surface facing the optical sensor array mounted on the platform. Movement of the sensor array over the patterned target surface corresponds to displacement of a catheter that is held in fixed relation to either the sensor array or the patterned target surface.

The type of optical sensor array and target surface differs in accordance with various embodiments of the present invention. Embodiments of the invention that utilize known optical mouse sensors generally rely upon diverse scatter patterns arising from physical variations in target surfaces that reflect light from a source onto the sensor array. However, in other embodiments of the invention the optical sensor array senses changes in the optical properties of the target surface (e.g., color or intensity). Such optical sensor arrays are especially useful in instances where the physical properties of the target surface do not sufficiently vary to render distinguishable scatter patterns.

In general, movement is sensed by correlating pattern images over time to detect physical shifts of the target surface. In an embodiment of the invention, since a catheter is allowed only one degree of freedom (linear movement), tracking need only take place in one dimension. However embodiments of the present invention rely upon sensors that operate in two dimensions. Sensing shifts in multiple dimensions is not precluded by the present invention.

The present invention is not limited to how a catheter is displaced in relation to the sensor array's field of view. The pull-back sensor measures actual catheter displacement and therefore does not require gear-driven pull-back devices such as the one described in Vujanic et. al. U.S. Pat. No. 6,290,675. However, the present invention is incorporated into such pull-back devices in alternative embodiments of the invention. Furthermore, the present invention can be advantageously incorporated into a pull-back device of the type utilized to displace a rotating crystal ultrasound transducer imaging catheter.

The present invention is not limited by the particular type of catheter device drawn through the optical sensor. While the present invention is preferably incorporated into a system that utilizes an array of ultrasound transducers to provide cross-sectional image data, the present invention is utilized to measure displacement of a variety of flexible elongate members including, without limitation, guide wire devices and treatment catheters (e.g., for performing balloon angioplasty and/or deploying a stent).

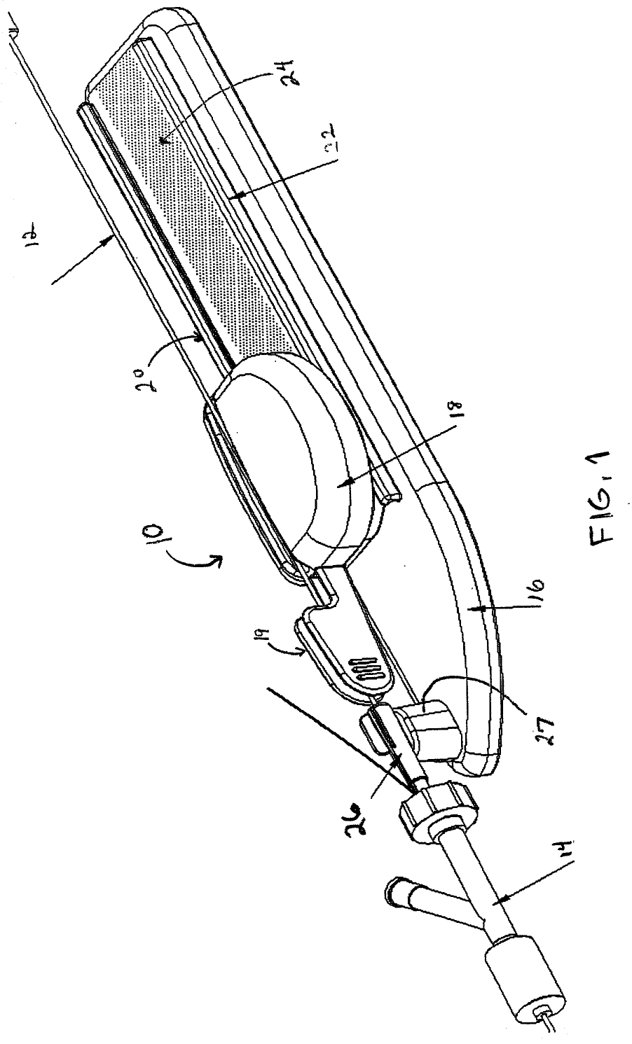

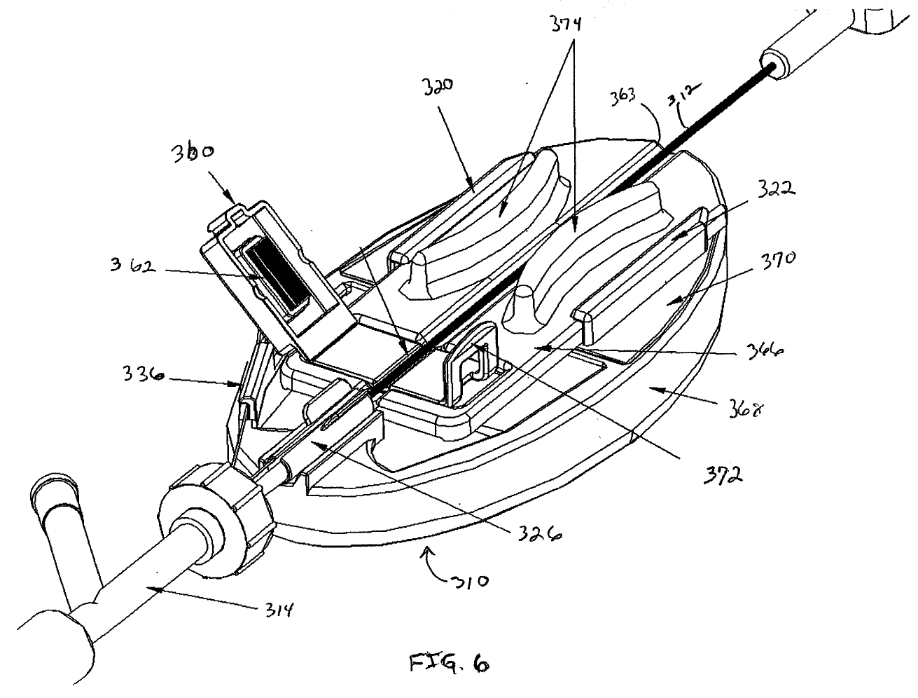

Turning to FIG. 1, an embodiment of the present invention is illustratively depicted. In particular, a wireless version of an optical pullback sensor assembly 10 is depicted. A catheter 12 is depicted in an operative arrangement wherein displacement of the catheter 12 in relation to a relatively statically positioned hemostatic valve 14 is tracked by the optical pullback sensor assembly 10. The optical pullback sensor assembly 10, in an embodiment of the present invention, includes a base 16 and a carriage 18. A catheter grip 19 extends from the carriage 18. The catheter 12 passes between the flexible pads of the grip 19 such that when the two pads are squeezed together the grip 19 engages the catheter 12 and facilitates coincidental movement of the catheter 12 and the carriage 18. The base 16 and carriage 18 are formed, by way of example, using known plastic injection molding techniques. In an embodiment of the invention the pullback assembly base is about 26.0 cm and has a pullback distance of approximately 15.0 cm. However, in alternative embodiments (see, e.g., FIGS. 6-9), the length and pullback distance are considerably shorter (e.g., 12.0 cm. length and 7.0 cm. pullback distance) Thus, the pullback device is both highly mobile and easily disposed after use. The carriage 18 is shaped/sized to fit comfortably within an adult hand. The electronics/processing circuitry of the pullback assembly 10 are of well-known variety and thus can be mass produced at relatively low cost using off-the-shelf components.

The carriage 18 underside (facing the base 16) is structurally configured to facilitate sliding engagement with the base 16 along a guide path. In the illustrative embodiment set forth in FIG. 1 such sliding engagement is achieved, for example, by a first guide 20 and second guide 22 that are integrally molded within the base 16. In alternative embodiments the guides are molded into the underside of the carriage 18, and a complementary, raised surface on the base 16 engages the guides on the carriage 18. In yet other embodiments, a monorail guides the carriage 18 along a path.

The optical pullback sensor assembly 10 utilizes an optical sensor array having a set of optical sensor cells and pattern recognition techniques that act upon the sensor cell output to detect displacement of the catheter 12 when placed in an operative position depicted in FIG. 1 and drawn coincidently with the carriage 18 over the base 16. In the illustrative embodiment of the invention depicted in FIG. 1, the catheter 12's displacement is indirectly measured by determining, from a pattern matching algorithm applied to frames of image data rendered by the optical sensor array, displacement of the carriage 18 in relation to the base 16. In the illustrated example, the base 16 includes a patterned target surface 24 that is sufficiently irregular such that movement of the target surface 24 across the field of view of the optical sensor array is detected to render a displacement measure. The patterned target surface 24 is textured to scatter reflected light. However, in an alternative embodiment visual artifacts (e.g., different colors/shades) are utilized in conjunction with a wide light source and sensor array that does not rely upon scattering to enhance image/displacement detection.

In the illustrative embodiment of FIG. 1, the carriage 18 houses a number of electronic components. The carriage 18 carries an optical sensor array as well as a coherent, collimated light source that emits light in the direction of the target surface 24 and is reflected toward the optical sensor array. The exemplary carriage 18 also includes processor electronics, preferably comprising an application-specific integrated circuit (ASIC) and associated known specific-purpose circuitry that fits within the limited space of the carriage 18. Such circuitry exists in current optical mouse technology that guide position of graphical user interface pointer. However, such circuitry is simplified in an embodiment of the invention wherein displacement is measured in a single dimension.

The optical pullback sensor assembly 10 is signally coupled to an imaging processor system that receives displacement information rendered by the processor electronics. The displacement information is thereafter correlated to cross-sectional image data acquired by the imaging system from an ultrasound probe disposed on a distal end of the catheter 12 and inserted into a vessel for diagnosis/treatment. In the wireless embodiment depicted in FIG. 1, a transmitter coupled to the processor electronics communicates a signal to a receiver/antenna coupled to the imaging processor system. Alternatively, a wire connection couples the pullback sensor assembly 10's processor electronics to the imaging process system. Furthermore, while the illustrative embodiment depicts the target surface 24 on the base 16, in a complimentary alternative embodiment of the invention the target surface 24 is located on the underside of the carriage 18 and the optical sensor array is located within the base 16. In such case, the electronics and signal transmission circuitry are housed in the base 16.

The catheter 12 passes through the hemostatic valve 14. The hemostatic valve 14 engages a hemostatic valve adaptor 26 mounted upon a support post 27 extending upwardly from the base 16. The hemostatic valve adaptor 26 facilitates maintaining the hemostatic valve 14 in a fixed physical relation to the base 16 as the catheter 12 is displaced within a body. The adaptor 26 is slotted to enable expansion to accommodate a range of hemostatic valve stem circumferences.

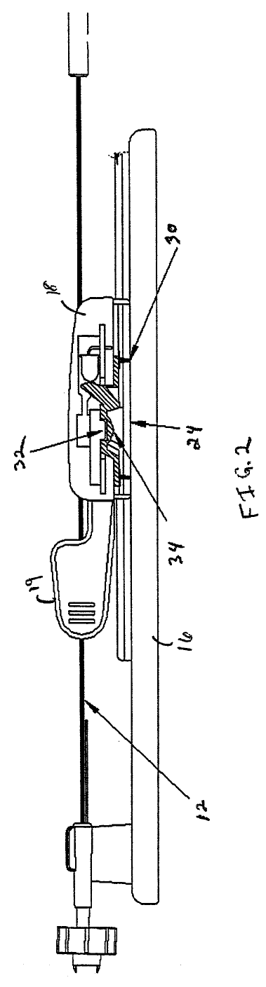

Turning to FIG. 2, a demonstrative illustration depicts a side cross-sectional view of the carriage 18 and base 16 depicted in FIG. 1. The sensor array data processing and communication electronics are of well-known composition and are therefore not addressed in detail. A wiper seal 30 is fitted to the surface of the carriage 18 facing the base 16. The wiper seal prevents fluids/debris from obscuring the target surface 24 pattern that facilitates detecting displacement of the carriage 18 in relation to the base 16. In an embodiment of the invention, the wiper seal 30 forms a perimeter around a field of view of an optical motion sensor 32 that includes both a light source and an optical sensor array. The wiper seal 30 also prevents foreign matter from obscuring a lens 34 that focuses light reflected from the target surface 24 onto the optical sensor array.

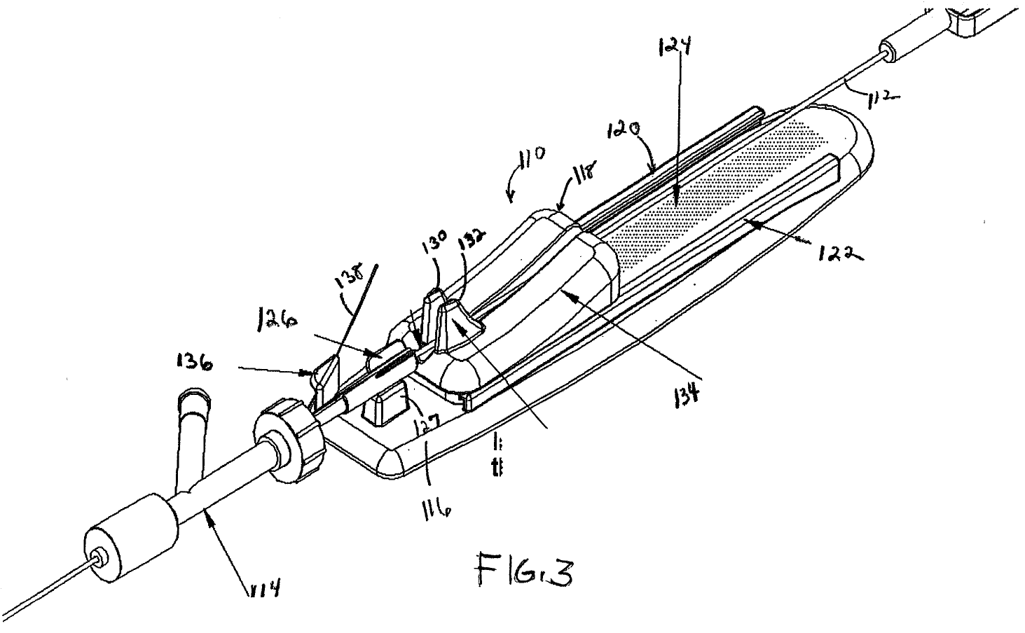

Turning to FIG. 3 an alternative embodiment of the optical pullback sensor assembly 10 depicted in FIG. 1 is depicted. An optical pullback sensor assembly 110 includes a base 116 and a carriage 118 that slides over a target surface 124 on the base 116. A hemostatic valve 114 connects to the base 116 via an adaptor 126 mounted upon a support post 127 extending upwardly from the base 116. Guides 120 and 122 confine the carriage 118 to a substantially linear sliding path defined by the guides 120 and 122 on the base 116. As in the embodiment depicted in FIG. 1, the light source, sensor array and image data processing electronics are housed within the carriage 118.

The embodiment depicted in FIG. 3 presents an alternative carriage 118 shape that enables a user to pinch a catheter 112 in the region of the carriage 118 between a pair of adjacent upward projections 130 and 132 from the upper surface of the carriage 118 and an upwardly sloping rear portion of the carriage 118. The projections 130 and 132 form a valley within which the catheter 112 sits. An upward sloping surface 134 provides better engagement of a user's hand as the catheter 112 is pulled back during an imaging procedure. A guide wire deflector 136 protrudes from the upper surface of the base 116 and separates a guide wire 138 from the catheter 112.

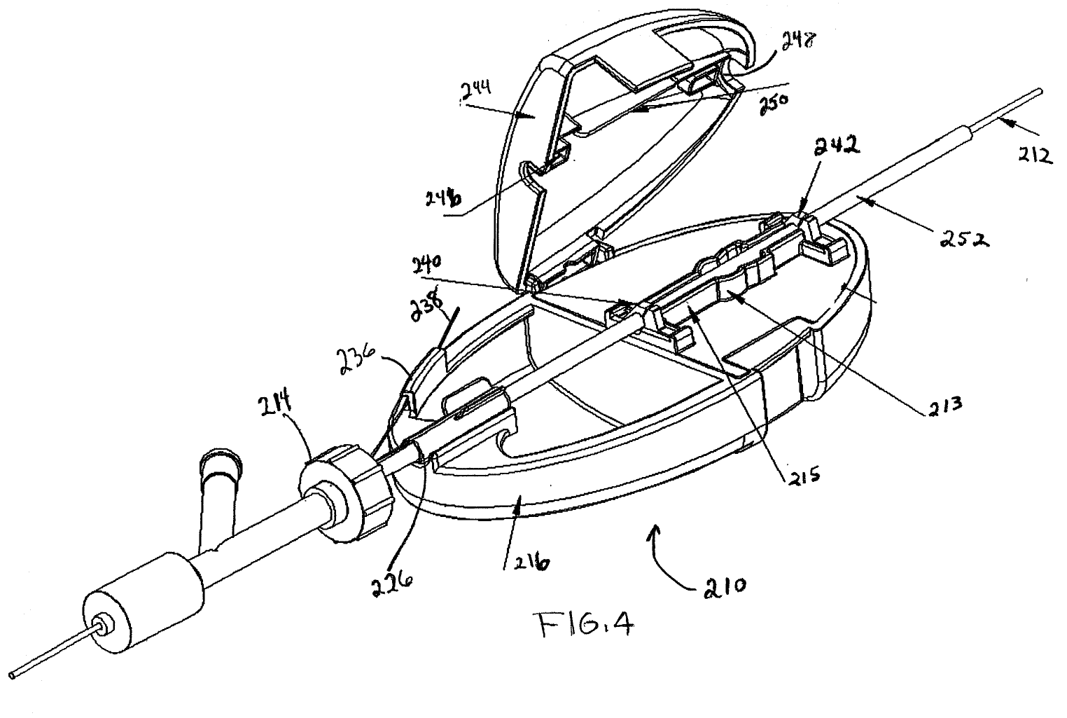

Having described embodiments of the invention wherein a target surface is provided by one of the sliding components (i.e., the base or the carriage) of the optical pullback assembly, an embodiment of the invention is depicted in FIG. 4 wherein a catheter 212 movement is directly sensed by a stationary optical sensor array. In contrast to the embodiments depicted in FIG. 1 and FIG. 3, an optical pullback sensor assembly 210 depicted in FIG. 4 does not include sliding components. Instead, the catheter 212 is drawn past a field of view of an optical sensor array mounted within the pullback sensor assembly 210 and facing the catheter 212 at position 213 of a groove 215 formed within a plastic molded base 216. A hemostatic valve adaptor 226 expands to receive and hold a hemostatic valve 214's stem. A guide wire deflector 236 separates a guide wire 238 from the catheter 212 to prevent the guide wire 238 from interfering with a catheter pullback.

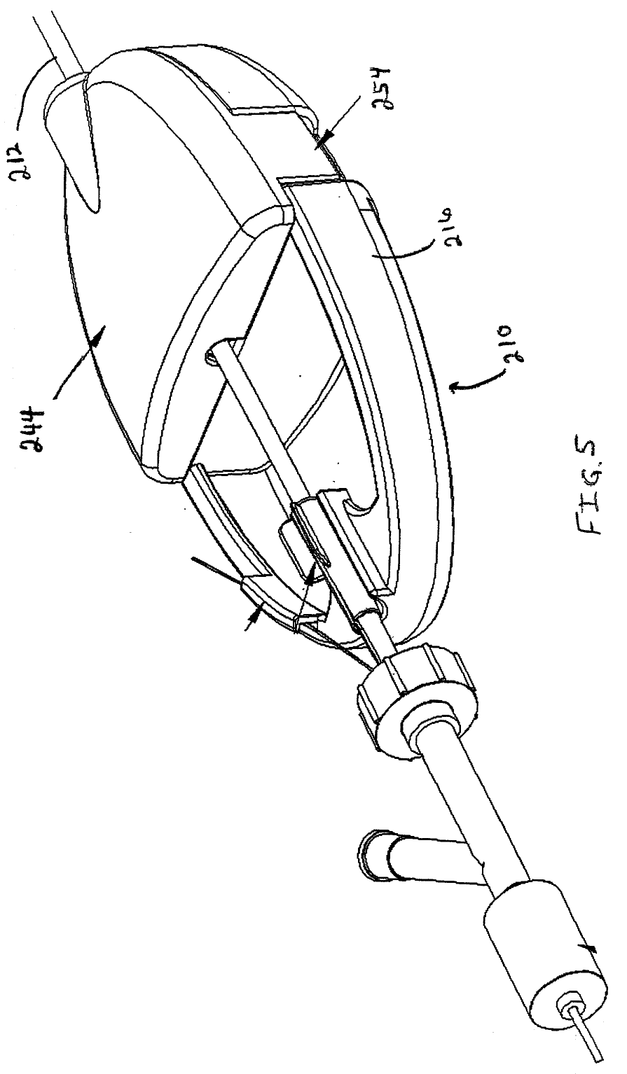

Certain additional features of the optical pullback sensor assembly 210 ensure the proper operation of the sensor assembly 210 in an operating room environment. Silicone seals 240 and 242 include a slot to enable a catheter to be snapped into place prior to closing a hinged cap 244 that includes a series of projections 246, 248 and 250 that ensure that the catheter passes through an intended field of view of the optical sensor array at position 213. Projections 246 and 248 abut the silicone seals 240 and 242 when the hinged cap 244 closes (as shown in FIG. 5) and thereby trap the catheter 212. The seals 240 and 242 also ensure that fluids and debris do not enter the groove 215 via the catheter 212 during a pullback procedure. The projection 250 confines the position of the catheter 212 when the hinged cap 244 closes.

It is also noted that in the embodiment depicted in FIG. 4 the circumference of the catheter 212 is increased by placing, for example, a sleeve 252 around the catheter 212. The increased circumference enhances the field of view occupied by the catheter 212 as it is drawn past the optical sensor array of the sensor assembly 210. In a particular embodiment, the sleeve 252 is a tube made from PVC heat shrink material. The sleeve 252 is intended to provide a more detectable surface area for tracking linear displacement of the catheter 212. Other exemplary modifications to enhance tracking include modifications to the surface of the catheter 212 itself, using a higher resolution sensor array, utilizing a different type of optical sensing mechanism (e.g., image pattern matching such as a color or shade matching algorithm rather than scatter patterns created by a textured surface).

Turning briefly to FIG. 5, the optical pullback sensor assembly 210 is depicted in its closed, operative position. The hinged cap 244 closes down upon the base 216 and a release latch 254 clamps upon a ridge on the side of the base 216. The catheter 212 passes through the pullback sensor assembly 210 (and past the enclosed optical sensor array) an amount corresponding to the displacement of a probe affixed to the distal end of the catheter 212.

Turning to FIG. 6, yet another embodiment of the present invention, a variation of the sliding carrier version depicted in FIG. 1, is depicted wherein the catheter 312 is held in place by a hinged lid 360 having a silicone pad 362. A hemostatic valve 314 frictionally engages an adaptor tube 326 and the catheter 312 is placed within a groove 363 on the upper surface of the optical pullback sensor assembly 310. A guide wire retainer 336 deflects the guide wire. A slider 366, restricted to linear movement by a set of rails 320 and 322, carries the hinged lid 360. Though not visible in the drawing, a light source and optical sensor array are housed within a main housing 368 (enclosed inpart by a housing cover 370).

When a user wishes to perform an actual pullback imaging procedure, the hinged lid 360 is closed and held by a hinge clip receptacle 372, the silicone pad engages and holds the catheter 312 as a user displaces the slider 366. A set of finger grips 374 provide a convenient protrusion enabling the user to engage and linear displace the slider 366 thereby displacing the catheter 312 by a corresponding amount. The displacement is sensed by the enclosed optical sensor array based upon variations sensed in light reflected by an underside surface of the slider 366 as the user displaces the slider in relation to the main housing 368 during a pullback procedure.

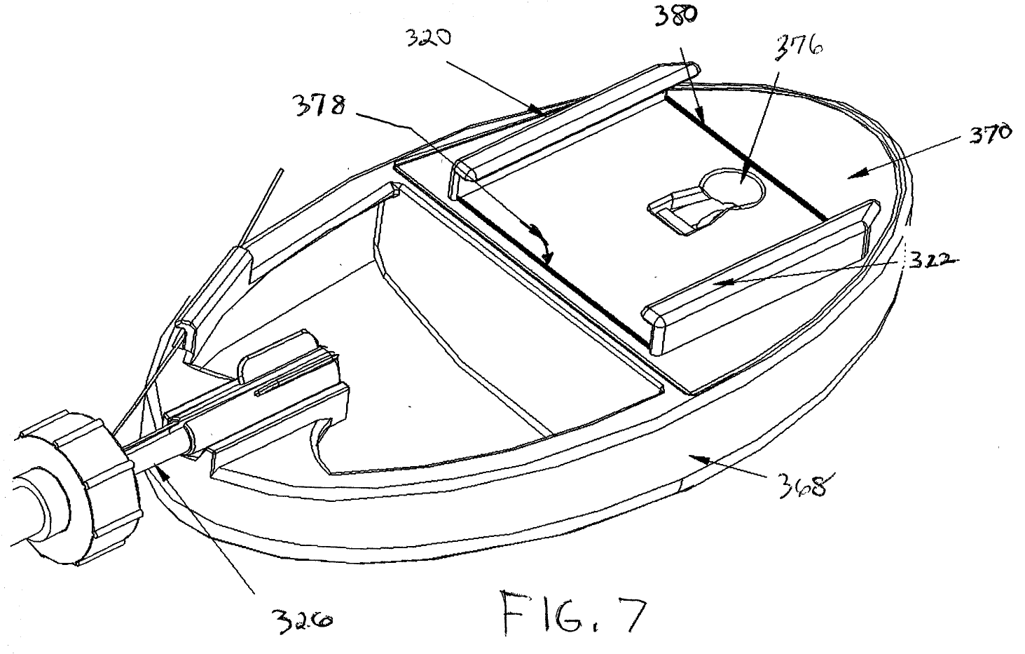

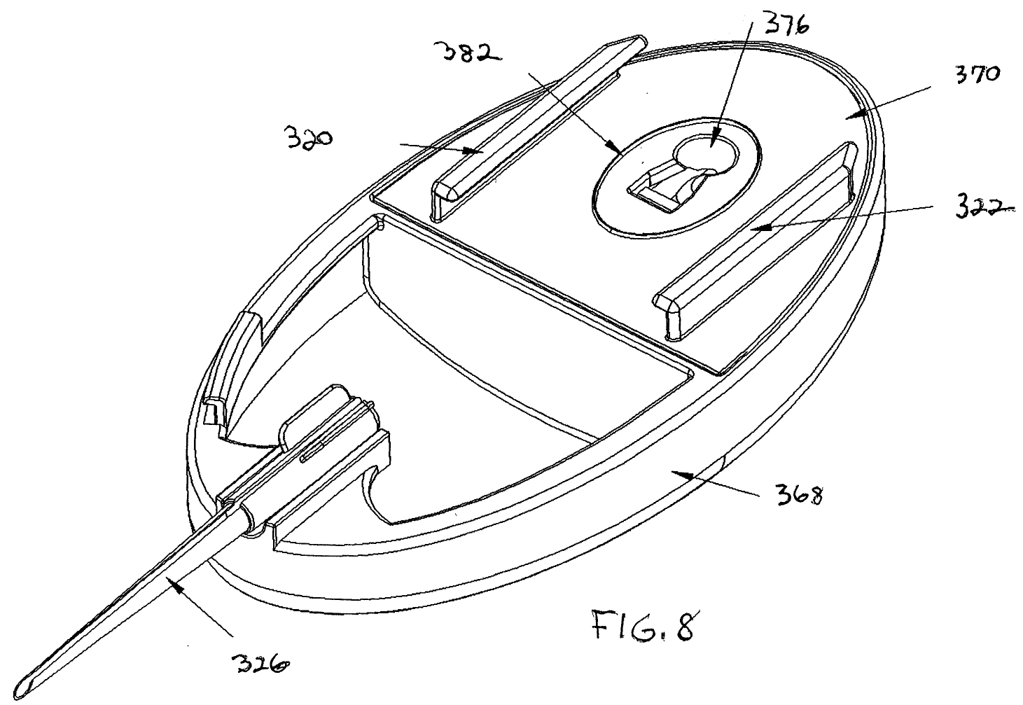

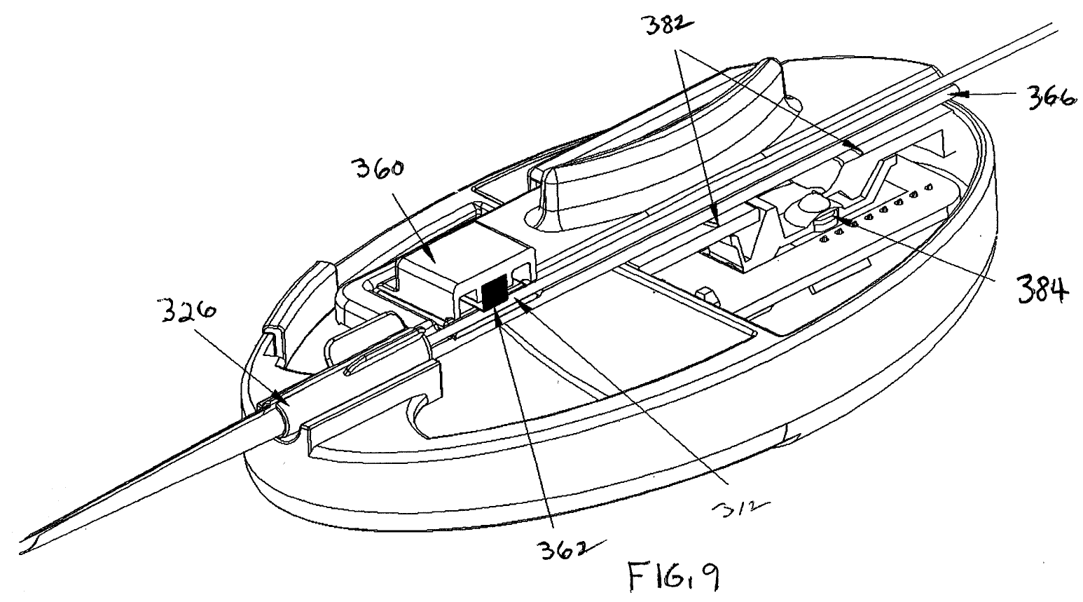

Turning briefly to FIGS. 7 and 8, a view is provided of the pullback sensor assembly 310 wherein the slider 366 has been removed to reveal an aperture 376 through which the underside of the slider 366 is viewed by an optical sensor array during a pullback procedure. Raised seals 378 and 380 in FIG. 7, and 382 in FIG. 8, prevent liquids from entering the cavity beneath the slider as well as shield the optical sensor from outside light. A cutaway view of the embodiment depicted in FIGS. 6, 7 and 8 is provided in FIG. 9, the cutaway view reveals the silicone pad 362 engaging the catheter 312. The view also depicts an optical sensor component 384 of the type typically used in optical mouse devices and arranged to sense light from a sensor light source deflected by the underside surface of the slider 366.

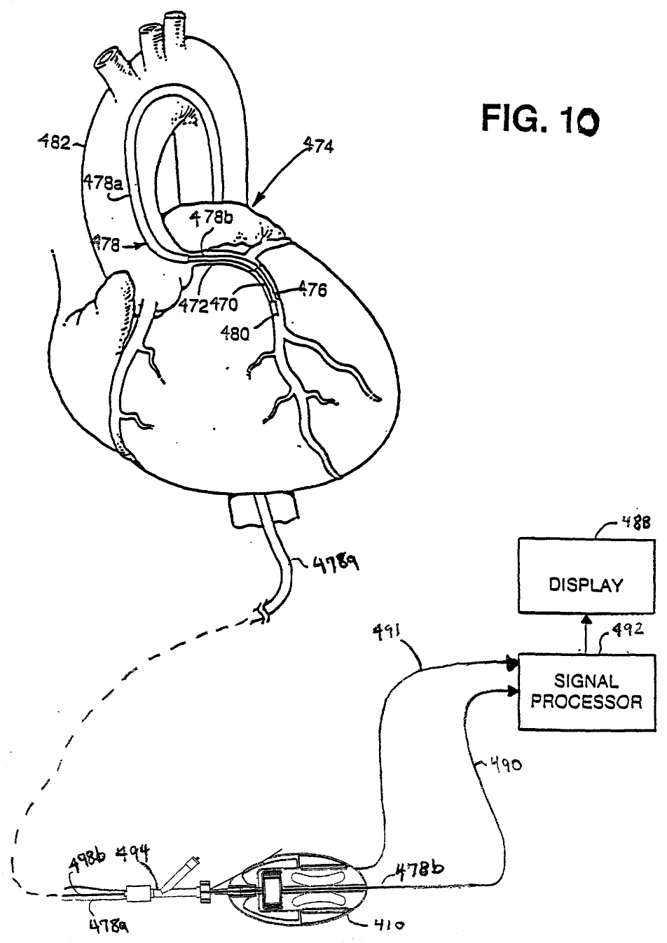

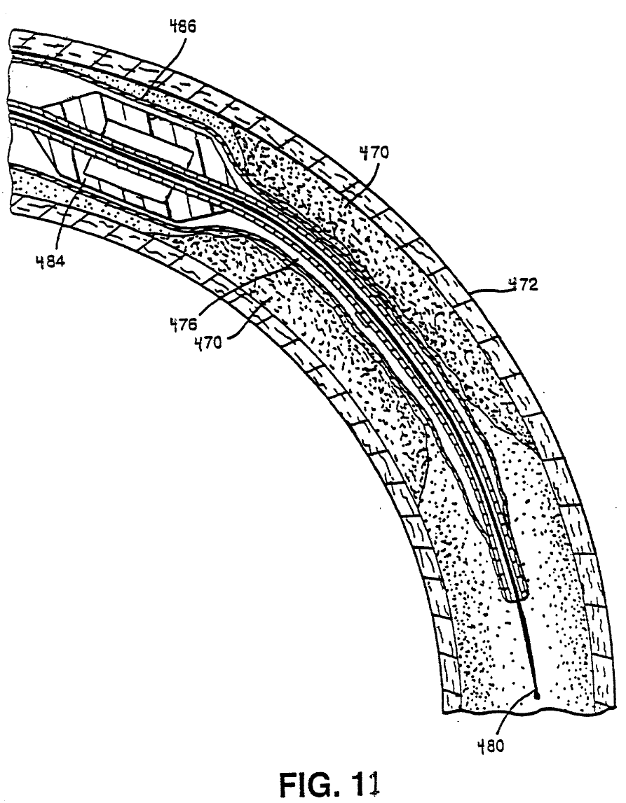

The above description has focused primarily upon the structure of the pullback sensor assembly in accordance with various exemplary embodiments of the invention. Turning now to FIGS. 10 and 11, an illustrative example of an environment and application of an intravascular ultrasound imaging system embodying the present invention is provided. A buildup of fatty material or plaque 470 in a coronary artery 472 of a heart 474 is treated by inserting a balloon 476, in a deflated state, into the artery 472 via a catheter assembly 478. In an exemplary catheter assembly arrangement illustrated in FIG. 10, the catheter assembly 478 includes a guide wire 480, a guide catheter 478a that threads through large arteries such as aorta 482, and a smaller diameter catheter 478b that fits within the lumen of the guide catheter 478a.

In the exemplary embodiment, after a surgeon directs the guide catheter 478a and the guide wire 480 through the aorta 482 to one of the coronary arteries, the smaller catheter 478b (carrying an ultrasound imaging device) is inserted within a coronary artery of interest. The guide wire 480 is initially inserted within the coronary artery past a portion that is blocked by plaque 470. In an embodiment where the catheter 478b includes a balloon 476, the catheter 478b is advanced to a portion of the artery 472 containing the plaque 470.

After the balloon 476 has entered the coronary artery 472, as in FIG. 11, an ultrasonic imaging device including a probe assembly 484 housed within the proximal sleeve 486 of the balloon 476 provides electrical imaging signals to a signal processor 492 via lines 490 enclosed within the catheter 478b. The processed electrical imaging signals provide a surgeon with a set of views of the artery on a display 488. In the illustrative embodiment, the transducers emit 20 MHz ultrasound excitation waveforms. However, other suitable excitation waveform frequencies are known to those skilled in the art. In operation, the transducers of the probe assembly 484 receive the reflected ultrasonic waveforms and convert the ultrasound echoes into electrical echo waveforms. The amplified electronic echo waveforms from the probe assembly 484, indicative of reflected ultrasonic waves, are transmitted via lines 490 to the signal processor 492 located outside the patient.

In an exemplary embodiment the guide catheter 478a couples to a two-port hemostatic valve 494. The stem of the hemostatic valve 494 engages an adaptor tube on the pullback sensor assembly 410. The catheter 478b passes through the two-port hemostatic valve 494 and through the pullback sensor assembly 410. In addition to the port through which the catheter 478b exits, the hemostatic valve 494 includes a port that may be used to perform an intravenous flush.

After placing a proximal portion of the catheter 478b within the optical pullback sensor assembly 410, displacement of the catheter 478b in the artery 472 is sensed by the optical pullback sensor assembly 410. The optical pullback sensor assembly 410 transmits a signal, representative of linear displacement of the catheter 478b, via line 491 to the signal processor 492. The signal processor 492 utilizes the displacement information received via line 491 and the imaging signals received via lines 490 to render a sequential or a composite image of the coronary artery 472 from a series of cross-sectional images obtained at various points of displacement along the length of the artery 472. In comparison to prior known arrangements, the optical pullback sensor assembly 410 and the disclosed exemplary embodiments offer an operationally simple arrangement for engaging the catheter 478b, drawing the catheter 478b backward, and tracking its linear displacement within a vessel during an intravascular ultrasound imaging procedure.

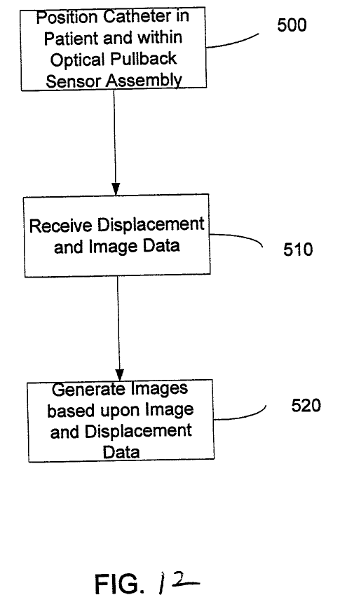

Turning now to FIG. 12, the following summarizes a set of steps for creating a composite image of a lumen and surrounding tissue from a set of cross-sectional images indexed according to linear displacement signals rendered by an optical pullback sensor assembly depicted, by way of example, in FIG. 1. Either before or after inserting the catheter 12 within a patient's body, during step 500 the catheter 12 is placed in engagement with the optical pullback sensor assembly 10. In embodiments such as the one depicted in FIG. 1 the catheter 12 is placed within a groove that facilitates reliably engaging the catheter 12 such that its displacement corresponds to relative displacement of a sliding surface/optical sensor pair. In other embodiments, such as the one depicted in FIG. 4, the groove guides the catheter 12 past an optical sensor array that facilitates measuring displacement of the catheter 12. In contrast to known systems that indirectly measure displacement according to rotation of drive axels/gears, the sensor assembly measures linear displacement of a surface that corresponds to the linear displacement of the catheter within the patients body.

Step 510 comprises a data input stage. In particular, during step 510 the catheter 12 supplies image data signals to an ultrasound image signal processor (e.g., signal processor 492). The signal processor also receives displacement data from the optical pullback sensor assembly 10. During step 510, the signal processor tags the input image and displacement data (e.g., applies a time stamp) to facilitate correlating the displacement data with the ultrasound image data. Thereafter, the time stamp data is used to match displacement and image signal values. Alternatively, the signal processor performs the correlation function by tagging image data with a current displacement value as the image signal data arrives at the signal processor.

During step 520 the ultrasound image signal processor renders images to be displayed on the display (e.g., display 486). For example, a stream of transverse cross-sectional images can be displayed sequentially according to their associated displacement positions. Alternatively, a longitudinal cross-sectional image is rendered by taking image data slices at each of a set of displacement positions and then juxtaposing them thereby rendering a particular longitudinal slice of an imaged vessel.

Illustrative embodiments of the present invention and certain variations thereof have been provided in the Figures and accompanying written description. Those skilled in the art will readily appreciate from the above disclosure that many variations to the disclosed embodiments are possible in alternative embodiments of the invention. Such modifications include, by way of example, modifications to the disclosed optical pullback sensor assembly structures, the optical sensor array type and displacement detection algorithms. The present invention is not intended to be limited to the disclosed embodiments. Rather the present invention is intended to cover the disclosed embodiments as well as others falling within the scope and spirit of the invention to the fullest extent permitted in view of this disclosure and the inventions defined by the claims appended herein below.

What is claimed is:

1. An optical pullback sensor assembly for measuring linear displacement of a flexible elongate member that is inserted within a body, the optical pullback sensor assembly comprising:
a light source;
a target surface that reflects at least a portion of light received from the light source;
an optical sensor array to receive at least a portion of the light reflected by the target surface; and
a tracking guide arranged such that displacement of the target surface in relation to the optical sensor represents displacement of the flexible elongate member.

2. The optical pullback sensor assembly of claim 1 wherein the optical sensor array is fixed in place with respect to a base and the target surface moves across the field of view of the optical sensor array.

3. The optical pullback sensor assembly of claim 2 wherein the target surface comprises a sleeve wrapped around the circumference of a segment of the flexible elongate member.

4. The optical pullback sensor assembly of claim 2 further comprising a seal through which the flexible elongate member passes prior to passing within the field of view of the optical sensor array.

5. The optical pullback sensor assembly of claim 2 wherein the tracking guide comprises a groove through which the target surface passes and thereby aligns the target surface within the field of view of the optical sensor array.

6. The optical pullback sensor assembly of claim 5 further comprising a hinged lid and wherein the hinged lid comprises one or more integrally molded confinement surfaces that restrict movement of the target surface to substantially linear displacement within the field of view of the optical sensor array when the hinged lid is placed in a closed position.

7. The optical pullback sensor assembly of claim 1 wherein the light source emits coherent light.

8. The optical pullback sensor assembly of claim 7 wherein the coherent light received by the target surface comprises collimated light.

9. The optical pullback sensor assembly of claim 1 wherein the target surface is textured to scatter the light reflected from the target surface and received by the optical sensor array.

10. The optical pullback sensor assembly of claim 1 further comprising a guide wire deflector.

11. The optical pullback sensor assembly of claim 1 further comprising a hemostatic valve adaptor for facilitating maintaining a hemostatic valve in a fixed physical relation to a base for the optical pullback sensor assembly as a flexible elongate member is displaced within a body.

12. The optical pullback sensor assembly of claim 1 wherein the target surface is fixed to a base and the field of view of the optical sensor array moves across the target surface.

13. The optical pullback sensor assembly of claim 12 wherein the target surface is a substantially flat, textured surface.

14. The optical pullback sensor assembly of claim 12 wherein the tracking guide comprises at least a first rail aligned with the direction of measured relative displacement between the optical sensor array and the target surface.

15. The optical pullback sensor assembly of claim 14 wherein the tracking guide comprises a second rail in parallel to the first rail.

16. The optical pullback sensor assembly of claim 14 further comprising a carriage guided along a path defined by the rail, and wherein the carriage includes structural components enabling engagement of the flexible elongate member to facilitate linearly displacing the flexible elongate member coincidentally with displacement of the carriage.

17. The optical pullback sensor assembly of claim 16 wherein the carriage includes a rest comprising first and second projections forming a passage through which the flexible elongate member feeds.

18. The optical pullback sensor assembly of claim 16 wherein the structural components enabling engagement of the flexible elongate member comprise a grip extending from the carriage.

19. The optical pullback sensor assembly of claim 16 wherein a wireless transmitter is located on the carriage for transmitting displacement information to a receiver associated with an image data processing unit.

20. The optical pullback sensor assembly of claim 1 further comprising a wireless transmitter for transmitting displacement information to a receiver associated with an image data processing unit.

21. The optical pullback sensor assembly of claim 1 wherein the optical pullback sensor array comprises a set of sensor cells.

22. The optical pullback sensor assembly of claim 1 wherein the optical pullback sensor array is disposed opposite the target surface.

23. An optical pullback sensor assembly for measuring linear displacement of a flexible elongate member that is inserted within a body, the optical pullback sensor assembly comprising:
a light source;
a target surface that reflects at least a portion of light received from the light source;
an optical pullback sensor array adapted to receive a portion of the light reflected by the target surface; and
a tracking guide fixed to the pullback sensor assembly such that relative displacement between the target surface and the optical pullback sensor array may be measured and corresponds to measured linear displacement of the flexible elongate member.

24. A method for rendering images from ultrasound image data provided by a probe attached to a flexible elongate member and from positional information provided by an optical pullback sensor assembly that measures linear displacement of the flexible elongate member, the method comprising the steps of:
obtaining ultrasound image data by the probe;
displacing the flexible elongate member;
sensing displacement of the flexible elongate member by an optical sensor arranged to receive light reflected from a target surface onto the optical sensor, thereby rendering positional information for the ultrasound image data;
transmitting the positional information to an image processor;
transmitting the ultrasound image data to the image processor;
correlating the positional information with ultrasound image data; and
rendering images based upon the correlated positional information and ultrasound image data.

25. The method of claim 24 wherein the sensing step is performed by an optical pullback sensor assembly comprising:
a light source;
a target surface that reflects at least a portion of light received from the light source;
an optical pullback sensor array for receiving a portion of the light reflected by the target surface; and
a tracking guide fixed to the pullback sensor assembly such that relative displacement between the target surface and the optical pullback sensor array may be measured and corresponds to measured linear displacement of the flexible elongate member.

26. The method of claim 25 wherein the transmitting the positional information step is carried out by communicatively coupling a displacement data signal output of the optical pullback sensor assembly, corresponding to displacement of the probe, to an image processor.

27. The method of claim 26 wherein the transmitting the ultrasound image data step is carried out by communicatively coupling an image signal output rendered by the probe to the image processor.

28. The method of claim 24 further comprising placing the flexible elongate member within an operative position with regard to the optical pullback sensor assembly by fixedly engaging the flexible elongate member to a slider on the pullback sensor assembly.

29. The method of claim 24 further comprising placing the flexible elongate member within an operative position with regard to the optical pullback sensor assembly by slidingly confining the flexible elongate member within a channel on the pullback sensor assembly, and wherein the channel is aligned with an aperture through which a portion of the light reflected by the flexible elongate member is received by the optical sensor array.

30. The method of claim 24 wherein the probe is disposed within a body lumen during the obtaining step.
